(19) United States
(12) Patent Application Publication
(10) Pub. No.: US 2025/0315897 A1
(43) Pub. Date: Oct. 9, 2025

(54) System and Method for Processing a Claim

(71) Applicant: Guardian Life Insurance Company of America, New York, NY (US)

(72) Inventors: Jessica Vanscavish, Hellertown, PA (US); Kausar Hasan, West Windsor Township, NJ (US); Dawn Sandt, Bethlehem, PA (US)

(73) Assignee: Guardian Life Insurance Company of America, New York, NY (US)

(21) Appl. No.: 19/171,099

(22) Filed: Apr. 4, 2025

Related U.S. Application Data
(60) Provisional application No. 63/574,411, filed on Apr. 4, 2024.

(51) Int. Cl. G06Q 40/08; G06F 21/31; G16H 10/60

(52) U.S. Cl. CPC G06Q 40/08; G06F 21/31; G16H 10/60

(57) ABSTRACT

A system and method for processing a claim includes a consumer opt-in module, a consumer verification module, a policy, a consumer electronic health record, an electronic data feed, a code catalogue comprising a first code and a second code, a notification module, an application programming interface configured to facilitate access to data of the consumer electronic health record, a large language model, and a database configured to receive queries, updates, and validations from the large language model. The system and method may further include an input form, a claim manager module, and a code library. An error detection module may be implemented to monitor the electronic data feed, identify errors, and release an error notification; and a control module configured to receive the error notification. The system and method may also classify a claim as payable or potentially payable.

### TECHNICAL FIELD

This disclosure relates to a system and method for processing a claim, and, more particularly, to a system and method for processing a claim pertinent to insurance, care, and/or disability through an automatic submission of the claim on behalf of a consumer or patient.

### BACKGROUND

Current systems and methods for processing a claim, which may be included in a variety of health-related or property-related settings, typically include claim submissions being manually submitted by, or on behalf of, a consumer or patient. Prior to manually submitting the claim, the submitter of the claim would have to understand whether the claim is likely to be approved. This would involve the need to further understand of the nature of the claim in relation to one or more policies. It is therefore critical for the claim submitter to be aware of what items or events would help them in determining what claim to submit and how to submit the claim.

Prior to this disclosure, traditional systems and methods for submitting a claim included a carrier consumer (e.g., an insurance carrier holder) or a doctor submitting a claim to an insurance carrier. Contents of the claim would be written on paper or electronically (e.g., on a computer or a mobile device). Whoever submitted the claim would have had to account for certain scenarios, certain codes, and certain arrangements, particularly if the claim concerns a plan holder with, for instance, multiple medical insurance carriers, multiple treating physicians, multiple facilities, etc. Some insurance carriers may have partnered with an entity which has built a data-matching computer program to identify potential claims for the insurance plan holder. This sort of partnership still involves manual reading and writing of claims, with the exception that the entity would spend time crafting claims instead of the consumer/insurance plan holder. Such a program is limited to group plan holders who self-insure the medical insurance.

Current, manual procedures for submitting claims are subpar. These procedures typically have, among other things, high risks of error, may require large amounts of effort, and result in unclaimed benefits. It is therefore desirable to develop a system and method that incorporates processing a claim through an automatic submission of the claim on behalf of a consumer or patient.

### SUMMARY OF THE DISCLOSURE

In one implementation, a system for processing a claim includes a consumer opt-in module; a consumer verification module; a policy; a consumer electronic health record; an electronic data feed; a code catalogue comprising a first code and a second code; a notification module; an application programming interface configured to facilitate access to data of the consumer electronic health record; a large language model configured to interact with the application programming interface, obtain the consumer electronic health record, read data of the consumer electronic health record, assign the first code to the data of the consumer electronic health record based on the consumer electronic health record's contents, read the electronic data feed, associate the data of the consumer electronic health record and the assigned code with one or more aspects of the electronic data feed to create an associated data set, read the policy, compare the associated data set with the policy, generate a claim based on the comparison between the associated data set and the policy, assign the second code to the generated claim, generate a notification via the notification module based on the second code, and notify whether the generated claim is accepted, under review, or denied; and a database configured to receive queries, updates, and validations from the large language model.

One or more of the following features may be included. The application programming interface may include an authentication module. The system may include an input form. The system may include a claim manager module. The claim manager module may be configured to generate the input form when the generated claim is under review. The generated claim may be automatically sent to the claim manager module if the generated claim is denied. The generated claim may be editable by an individual. The database may include a code library and a health data standard, wherein the code library is configured to interact with the health data standard and wherein the large language model is configured to access and employ the code library. The electronic data feed may include claim adjudication data. The system may include an error detection module configured to monitor the electronic data feed, identify errors, and release an error notification; and a control module configured to receive the error notification.

In another implementation, a method for processing a claim includes opting in a consumer; verifying the identity of the consumer; obtaining a policy of the consumer; obtaining the consumer's electronic health record; providing an electronic data feed; providing a code catalogue comprising a first code and a second code; providing a notification module; providing an application programming interface configured to facilitate access to consumer's electronic health record; providing a large language model configured to interact with the application programming interface, obtain the consumer electronic health record, read data of the consumer electronic health record, assign the first code to the data of the consumer electronic health record based on the consumer electronic health record's contents, read the electronic data feed, associate the data of the consumer electronic health record and the assigned code with one or more aspects of the electronic data feed to create an associated data set, read the policy, compare the associated data set with the policy, generate a claim based on the comparison between the associated data set and the policy, assign the second code to the generated claim, generate a notification via the notification module based on the second code, and notify whether the generated claim is accepted, under review, or denied; and sending queries, updates, and validations to a database.

One or more of the following features may be included. The method may include sending the generated claim to a claim manager module. The method may include investigating the generated claim and notifying the consumer of investigation details. The method may include obtaining raw data from a carrier. The method may include filtering the raw data. The method may include generating claims data arranged to be fed into the large language model. The method may include obtaining biometric data of the consumer. The method may include the first code and the second code each being a healthcare code or a medical code. The method may include extracting health data of the consumer after generating a claim. The method may include classifying the claim as payable or potentially payable.

### BRIEF DESCRIPTION OF THE DRAWINGS

Various embodiments in accordance with the present disclosure will be described with reference to the drawings in which:

FIG. 1 illustrates an example of a system for processing a claim.

FIG. 2 illustrates an example of a consumer opt-in module.

FIG. 3 illustrates an example of a payable claim and a potentially payable claim being generated by a large language model.

FIG. 4 illustrates an example of a verification module.

FIG. 5 illustrates an example of a claim manager module.

FIG. 6 illustrates an example of a large language model architecture.

### DETAILED DESCRIPTION OF THE PREFERRED EMBODIMENTS

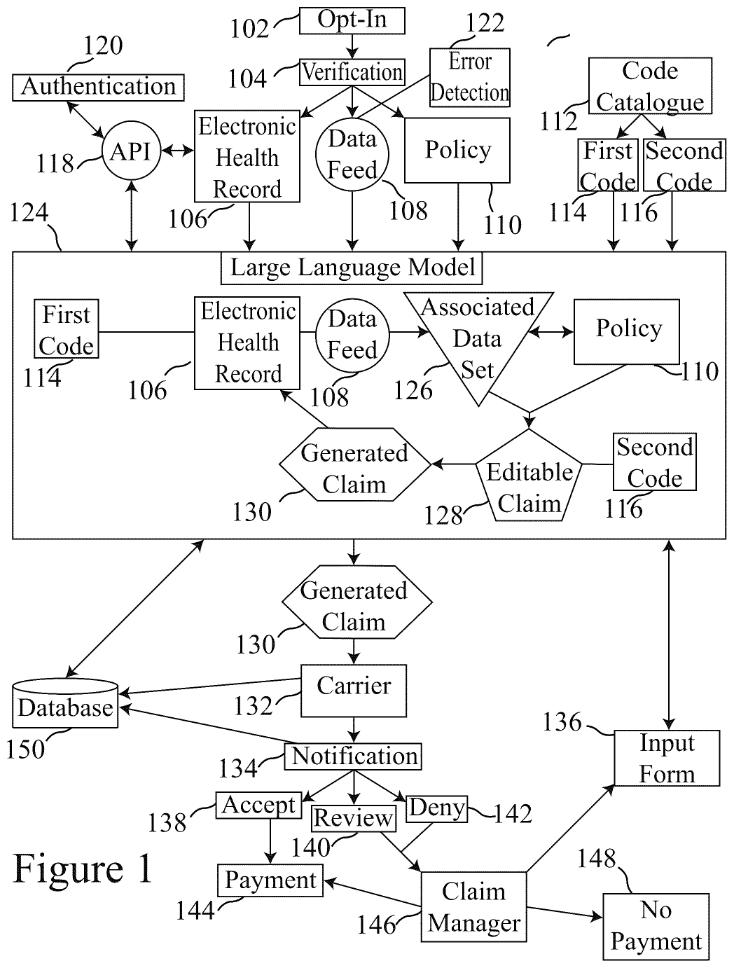

Referring to FIG. 1, there is shown an example of a system 100 for processing a claim. System 100 may include a consumer (or user) opt-in module 102, with which a consumer (or user) of system 100 may opt in to one or more options of system 100. For example, the consumer may use opt-in module 102 to participate in automatic payment mechanisms when enrolling for insurance and/or participate directly with a carrier 132 (e.g., an insurance carrier, a disability carrier, an indemnity carrier, a long-term care carrier, etc.) in choosing communication preferences and payment preferences. The consumer may use opt-in module 102 to authorize extracting and verifying data related to their healthcare, their health record, their disability record, their insurance records, and/or any records pertinent to the consumer's well-being. The consumer may use opt-in module 102 to consent that data, including their personal data and data related to their visits to, for example, a doctor, hospital, an inpatient or outpatient treatment facility, a treatment provider, an ambulance setting, and/or a medication provider. The consumer data may be used to automatically submit the claim on their behalf. Opt-in module 102 may include, without limitation, forms for the consumer to read and sign, an explanation of data to be accessed and used (e.g., medical records, insurance policy details, etc.), information on how data will be securely stored and transmitted, details on the type(s) of claim(s) which can be submitted, options for users to select which service(s) they consent to for automatic submission(s), an explanation of benefits and risks, user control options (including how to opt-out or modify their consent), contact information for support and queries, privacy policy and compliance statements, or a confirmation step where the user actively agrees to terms. Opt-in module 102 may further include a user authentication mechanism to ensure that the individual using opt-in module has authority to participate and/or consent to particular preferences and/or data usage methods.

Through opt-in module 102, system 100 may obtain data sufficient to identify the individual consumer. A consumer verification module 104 may verify the identity of the individual consumer. Verification means of verification module 104 may include multi-factor authentication, knowledge-based authentication, digital certificates, digital signatures, biometric verification, and authenticating a government identification document (e.g., passport, state ID, driver's license, etc.). Verification module 104 may alert the consumer of requiring additional information to ensure sufficient verification of the consumer (e.g., if an identification document is outdated or not readable, the verification module 104 may request an updated or readable document of the consumer). Additional information, such as caregiver or guardian information, may also be presented to verification module 104 so that a caregiver or a guardian may be verified and/or approved to participate in system 100 on behalf of the consumer, particularly if the consumer is unable to independently use opt-in module 102.

With the consumer's identity verified, one or more of the consumer's electronic health records 106 may be downloaded and/or extracted in system 100. One or more of the electronic health records may include the following information of the consumer: personal information, medical history, medication information, immunization records, lab test results, radiology images, vital signs, progress notes, treatment plans, insurance information, advance directives, appointment information, features for direct communication between patients and healthcare/wellness providers, prescription orders, reminders for preventive care, social determinants of health, patient-reported outcome measures, genetic information, behavioral and mental health records, telehealth consultation notes, patient preferences, care coordination notes, end-of-life planning, patient portal access logs, compliance and consent documentation, medical trial records, etc.

System 100 may further include an electronic data feed 108. Data feed 108 may include information of electronic health record 106, plus, for example, patient/consumer demographics, provider notes, procedures, encounter information, insurance claims data, appointment scheduling, discharge summaries, telehealth data, real-time monitoring data, public health notifications, insurance claim updates, pharmacy benefits data, healthcare supply chain information, supply chain information pertinent to products of a wellness industry, market and pharmaceutical updates, environmental and community health data, etc. Data feed 108 may provide a dynamic and comprehensive view of information relevant to healthcare delivery, disabilities, long-term care, and/or patient care management which extends beyond a scope of electronic health record 106. Data feed 108 may also include claim adjudication data for the consumers' past claims and/or claims from other scenarios resembling scenarios of the consumer.

As data feed 108 may include large amounts of data, it is possible that data feed 108 may have errors stemming from clerical errors and/or electronic bugs. An error detection module 122 may be employed in system 100 to monitor data feed 108. Error detection module 122 may also identify an error in data feed 108 and notify the consumer and/or a system administrator of the error. Error detection module 122 may also halt operations of system 100 if the error detection module 122 finds a significant error. A significant error may be pre-defined by the system administrator or by an industry standard.

A policy 110 of the consumer may be downloaded or extracted in system 100. Policy 110 may be a third-party policy imported into system 100. Policy 110 may be generated by system 100. Policy 110 may, for example, be a text document, a document in Portable Document Format (“PDF”), a Word document (“.docx”), an HTML document, an electronic publication, an XML document, an email, a digital form, or an Electronic Data Interchange document. Policy 110 may include, without limitation, a policy number, an effective date, premium details, policyholder information of the consumer, coverage benefits, exclusions and limitations, deductibles, co-payments and coinsurance, out-of-pocket maximums, network information, claim submission guidelines, prior authorization requirements, an appeals process, renewal and cancellation terms, contact information, etc.

System 100 may include a code catalogue 112 containing, for example, standardized codes for categorizing and coding various aspects of healthcare and/or wellness, including diagnoses, procedures, medical services, and equipment. Code catalogue 112 may be an electronic catalogue containing one or more standardized coding systems. The coding systems may include the International Classification of Diseases (“ICD”) coding system; the Current Procedural Terminology (“CPT”) coding system; the Healthcare Common Procedure Coding System (“HCPCS”); National Drug Codes (“NDC”); or Logical Observation Identifiers Names and Codes (“LOINC”). These coding systems ensure uniformity and accuracy in the documentation, billing, and reimbursement of healthcare/wellness services, facilitating efficient healthcare/wellness delivery and healthcare/wellness data analytics. System 100 may access code catalogue 112 via an electronic storage mechanism (e.g., local storage and/or cloud storage) and/or a download mechanism (e.g., a downloadable source). A first code 114 and a second code 116 may be extracted from code catalogue 112 during operation of system 100. First code 114 and second code 116 may be used to process billing across healthcare/wellness providers and insurers. First code 114 and second code 116 may be used to identify a payable claim. First code 114 and second code 116 may facilitate matching an insured product with a claim. First code 114 and second code 116 may be used to verify medical necessity of procedures; to detail specific services provided so as to allow an insurer of the claim to match the codes against covered services of, for example, policy 110; to verify whether procedures were performed in-network; to verify if pre-authorization requirements were met; or to ensure that a prescribed drug is covered under, for example, policy 110. Code catalogue 112 may facilitate accurate determination of eligibility of a claim for a diagnostic procedure, a surgery, a medication, etc. If all codes align with and are satisfied under covered services in, for example, policy 110 (e.g., deductibles, out-of-pocket maximums, copayments, etc.), the claim would be approved and/or processed for payment.

Data and/or information of electronic health record 106, data feed 108, policy 110, and code catalogue 112 may be read, obtained, or ingested by a large language model 124. Large language model 124 may be capable of reading plain text files, document formats, spreadsheet formats, presentation formats, email formats, programming languages, markup languages, rich text formats, or markdown formats. Large language model 124 may be an advanced type of artificial intelligence algorithm designed to understand, generate, and interact with human language. Large language model 124 may be pre-trained by other healthcare/wellness data and/or data of electronic health record 106, data feed 108, policy 110, and code catalogue 112. Large language model 124 may be a third-party large language model or a large language model developed specifically for system 100. Large language model 124 may read texts, code, photographs, data, videos, simulations, images, or pictures. Large language model 124 may generate text, code, videos, images, data, simulations, or pictures. Large language model 124 may use statistical patterns to predict words or tokens in crafting responses to prompts and/or authoring text based on input or information large language model 124 receives.

Large language model 124 may interact with an application programming interface (“API”) 118. API 118 may facilitate access to data of the consumer electronic health record. API 118 may further include an authentication module 120. Authentication module 120 may control authentication mechanisms of API 118. For instance, authentication module 120 may employ API keys or OAuth tokens to ensure that only authorized users or entities can access API 118 and/or the data API 118 provides. Large language model 124 may be enabled by authentication module 120 to interact with API 118 to obtain information of electronic health record 106. Large language model 124 may get information of electronic health record 106 via API 118.

Large language model 124 may read data of electronic health record 106 and assign first code 114 to electronic health record 106. Large language model 124 may include an event subscriber (not shown) to listen for events of electronic health record 106 (for example, an addition of new information from a recent accident or a recent visit to a healthcare facility). Event subscriber may be a separate, stand-alone large language model, or a third-party subscription service. Large language model 124 may also obtain contract version data from a carrier (e.g., carrier 132 in system 100) or electronic health record 106. Contract version data may include effective dates of policy coverage, coverage details, endorsements or riders, etc. Large language model 124 may assign first code 114 based on data of electronic health record 106 and/or the reading of electronic health record 106 by large language model 124. For instance, first code 114 may be based on information, such as a diagnosis, a performed medical procedure, a performed medical service, or a combination thereof, contained in electronic health record 106. First code 114 may be based on most-recent entries in electronic health record 106.

Large language model 124 may read data feed 108. Large language model 124 may listen for medical events from data feed 108. Large language model 124 may listen for medical events from data feed 108 by matching codes with consumer information. Large language model 124 may associate and/or combine information and its readings of both electronic health record 106 and data feed 108 to create an associated data set 126. That is, associated data set 126 may contain information from both electronic health record 106 and data feed 108. Associated data set 126 may contain information pertinent to the consumer. Large language model 124 may compare contents of associated data set 126 with contents of policy 110 to determine whether a claim could be payable under policy 110. Comparison of may include statistical analyses. The statistical analyses may depend on contents of electronic health record 106 or data feed 108 or a third-party source of healthcare/wellness data. Comparison may include matching a portion of policy 110 with a portion of associated data set 126 to facilitate generating a claim.

Large language model 124, following comparison and analyses of associated data set 126 and policy 110, may generate an editable claim 128. Large language model 124 may assign second code 116 to editable claim 128 based on content of editable claim 128. Second code 116 may, for instance, facilitate summarizing procedures, diagnoses, services, etc. contained in editable claim 128. Moreover, editable claim 128 may manually edited by the consumer, or on behalf of the consumer. Editable claim 128 allows the consumer or the caregiver to customize or tailor a claim which they believe should be submitted to carrier 132 or to another party. Should editable claim 128 be edited by the consumer or on behalf of the consumer, second code 116 may be changed to be consistent with the amended contents of editable claim 128. Editable claim 128 may be left as initially generated by large language model 124.

If the consumer, or someone on behalf of the consumer, finds editable claim 128 to be acceptable to them, then large language model 124 renders editable claim to a non-editable, generated claim 130. Generated claim 130 may subsequently be made a part of electronic health record 106. Generated claim 130 may also then be submitted to carrier 132 or to another party. Generated claim 130 may include dollar amounts requested to be paid by carrier 132 or another party.

Carrier 132 may then evaluate generated claim 130 either through their own models or through their own personnel. Carrier 132 may base their decision, in whole or in part, on second code 116 to accept (numeral 138), review (numeral 140), or deny (numeral 142) generated claim 130. Carrier 132 may then use notification module 134 of system 100 to generate an acceptance notification (numeral 138), review (numeral 140), or deny (numeral 142) generated claim 130. Should carrier 132 accept generated claim 130, carrier 132 may arrange for payment (numeral 144) of generated claim 130. Payment by carrier 132 may be in the full amount requested by generated claim 130 or in a partial amount requested. Payment may be consistent with preferences of the consumer set at consumer opt-in module 102. Should generated claim 130 be in review status or denied, generated claim 130 may be sent to a claim manager module 146.

Claim manager module 146 may further review (numeral 140) generated claim 130 by generating an input form 136 to be completed by the consumer or on behalf of the consumer. Input form 136 may be generated independently by claim manager module 146 or with assistance from large language model 124. Large language model 124 may interact with input form 136 to ingest inputted information and/or augment input form 136 to request more information from the consumer.

Input form 136 may require more information from a variety of sources or for a variety of purposes. Contents of input form 136 may be based on policy 110 and/or items desired by claim manager module 146. Input form 136 may request detailed medical records; proof of pre-authorization or referral; explanation of benefits (e.g., in case of dual coverage to determine coordination of benefits); itemized bills; accident reports; pharmacy prescription records; clarification of coding (e.g., clarification of first code 114 and/or second code 116); proof of eligibility; details of other insurance coverage; or supporting documentation for special circumstances. If input form 136 is completed by or on behalf of the consumer, contents of input form 136 may be submitted to large language model 124. Large language model 124 may then review generated claim 130 in light of new items from input form 136. Large language model 124 may also review electronic health record 106, data feed 108, and new content from input form 136 to change contents of or add contents to associated data set 126. Large language model 124 may then re-compare associated data set 126 with policy 110 to generate a new editable claim (not shown). The new editable claim may be edited in substantially the same way as editable claim 128. The new editable claim may then be finalized into a new generated claim (not shown) and be prepared for submission to carrier 132 for review. Carrier 132 may then decide to accept, further review, or deny the new generated claim.

Should generated claim 130 be denied (numeral 142), generated claim 130 may then be sent to claim manager module 146 for review. Claim manager module 146 may either finalize the denial by issuing a no payment decision (numeral 148) or render generated claim 130 to a reviewable status by seeking additional information via input form 136. That is, should carrier 132 deny generated claim 130, claim manager module 146 may ensure at least a second review of generated claim 130 via claim manager module 146.

A database 150 may be employed in system 100 to dynamically record and/or store some or all information of large language model 124 and of notification module 134. Large language model 124 may retrieve information from database 150 to update generated claim 130. Large language model 124 may submit drafts of generated claim 130 to database 150 for storage and/or validation purposes. Large language model 124 may query database 150. Database 150 may receive additional information from a source outside of system 100.

Database 150 may receive information or data from carrier 132. Large language model 124 may access information of carrier 132 via database 150. Notification 134 generated via carrier 132 may be sent to database 150 from which large language model 124 may access information of notification 134. Database 150 may receive raw data from carrier 132 or data that has been through a modification process.

Database 150 may include a code library and a health data standard (not shown). The code library may be configured to interact with the health data standard. The code library may be accessible to large language model 124. The code library and health data standard may ensure that software applications can accurately and efficiently process, interpret, and communicate health information according to widely accepted protocols. They may also facilitate interoperability between different healthcare and/or wellness systems, applications, and services, enhancing data exchange and integration across the healthcare and/or wellness ecosystem. Large language model 124 may employ the code library and the health data standard to assist in automated coding; claims validation; decision support; and fraud detection.

System 100 may have automated steps or procedures. For example, after the consumer opts in to have their data automatically extracted and processed in system 100 (e.g., numeral 102), subsequent modules and large language model 124 may automatically begin operating. Large language model 124 may also automatically generate a claim anytime electronic health record 106, data feed 108, or policy 110 are updated.

System 100, in addition to API 118 and other exemplary application programming interfaces of the foregoing disclosure, may include additional, various application programming interfaces (not shown). These additional application programming interfaces may provide protocols and tools for querying or retrieving information from one dataset to another dataset. For example, they could be used to query a data repository (e.g., database 150) or retrieve specific datasets or information needed by, for example, large language model 124. They may also handle requests by fetching data or formatting data for use (e.g., by large language model 124). These application programming interfaces may also facilitate real-time data exchanges; implement security measures; enable automation of data flows between datasets; or facilitate training of, for example, large language model 124.

System 100 may function within a mobile device (e.g., a smartphone, a mobile phone, a tablet, a laptop, etc.), a desktop or tabletop device (e.g., a personal desktop computer, a television, a touchscreen monitor, etc.), or any other electronic interface which a consumer may access or interact with (e.g., a program with access to data from a microphone or a smart-home setup).

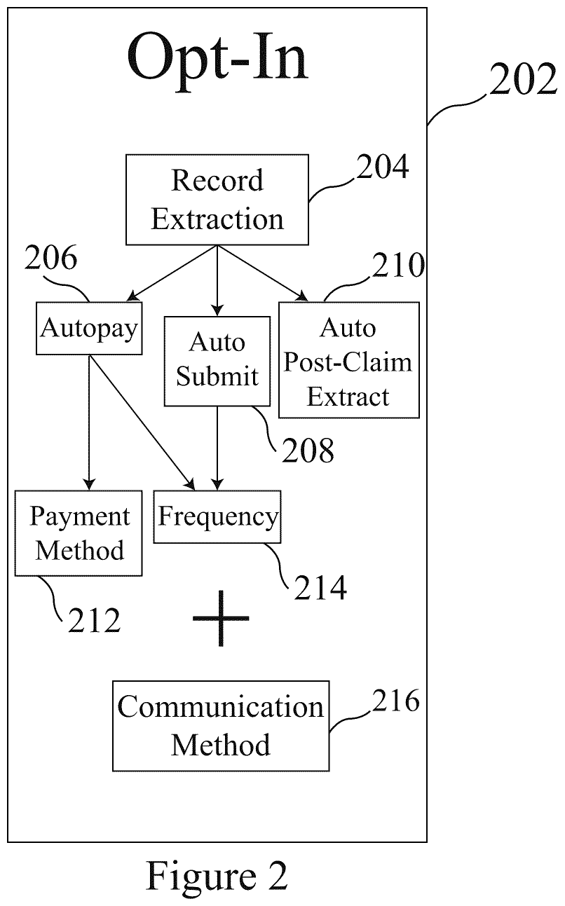

Referring to FIG. 2, there is shown an example of a consumer opt-in module 202. Opt-in module may be implemented in place of consumer opt-in module 102 as shown in FIG. 1. Opt-in module 202 may include presenting the consumer with a record extraction opt-in option 204, in which the consumer may authorize automatic health record data extraction. Record extraction opt-in option 204 may further present the consumer with types of record extraction, including extraction of payment details for automatic payments of partially paid claims (numeral 206). The consumer may set a preferred payment method 212 and/or identify the frequency for autopayment (numeral 214). Record extraction opt-in option 204 may show the consumer an auto-submit option 208, in which the consumer would authorize automatic submission of claims generated within system 100 to the consumer's carrier (e.g., insurance carrier). The consumer, like for autopayment, may identify the frequency for automatic submission of claims to their carrier (e.g., once a week, once a month, etc.). A third option which may be available to the consumer within record extraction opt-in option 204 is automatic data extraction following submission of a claim (“Auto Post-Claim Extract,” as shown at numeral 210). If the consumer opts into Auto Post-Claim Extract 210, the consumer may authorize system 100 to automatically extract additional health data of the consumer following submission of generated claim 130. This would provide control to the consumer as to what additional healthcare/wellness data system 100 would have access to following submission of generated claim 130 to the consumer's carrier. Opt-in module 202 may also inquire the consumer to identify the consumer's preferred communication method 216 (e.g., an application portal, mail, email, text, phone call, etc.). Opt-in module 202 may further include one or more application programming interfaces (not shown) for the consumer to opt-in via a carrier (e.g., an insurance carrier). One or more of the application programming interfaces may facilitate movement of opt-in information in a system like system 100. For instance, movement of opt-in information may include transferring opt-in information to verification module 104.

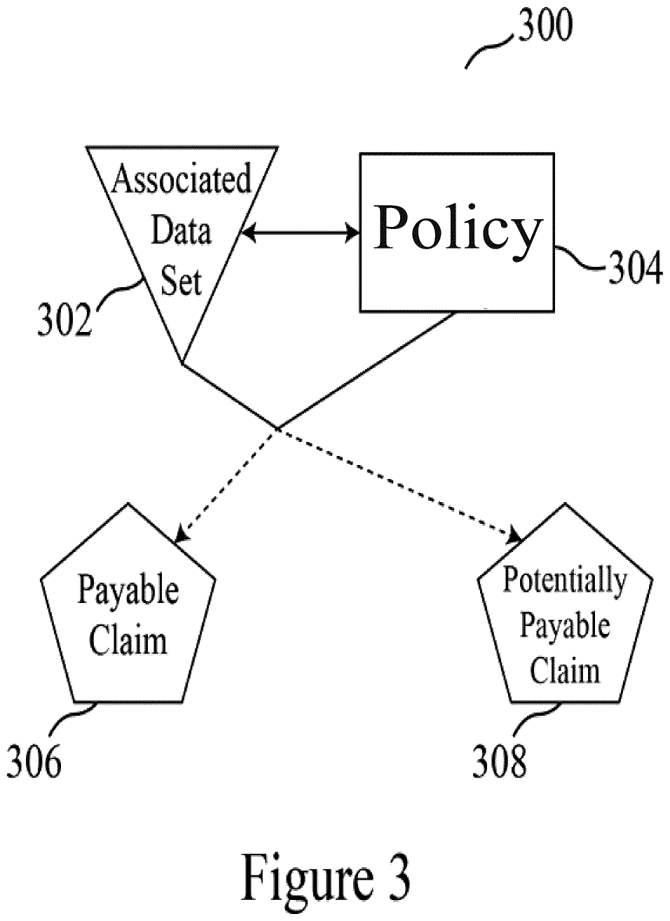

Referring to FIG. 3, there is shown an example of a large language model 300 which may be a part of system 100 or a part of another system. Large language model 300 may take an associated data set 302 (which may be substantially similar to associated data set 126 as shown in FIG. 1) and compare it with a policy 304. Depending on the contents of associated data set 302 and policy 304, large language model 300 may determine that associated data set 302 has sufficient information to generate either a payable claim 306 or a potentially payable claim 308. Payable claim 306 may be generated following a match of codes to an insured product. Payable claim 306 may lead to payment substantially similar to the scenario for payment as shown in FIG. 1 (numeral 144). Potentially payable claim 308 may result from large language model 300 needing more information about the consumer. Should a potentially payable claim 308 be generated, a trigger notice (not shown) may be sent to a claim manager module (not shown). The claim manager module may be substantially similar to claim manager module 146 in FIG. 1. With the information of potentially payable claim 308 at hand, the claim manager module may then validate eligibility and trigger a notice to a clearing house (not shown) to do a full extract of medical records to determine if the claim is payable.

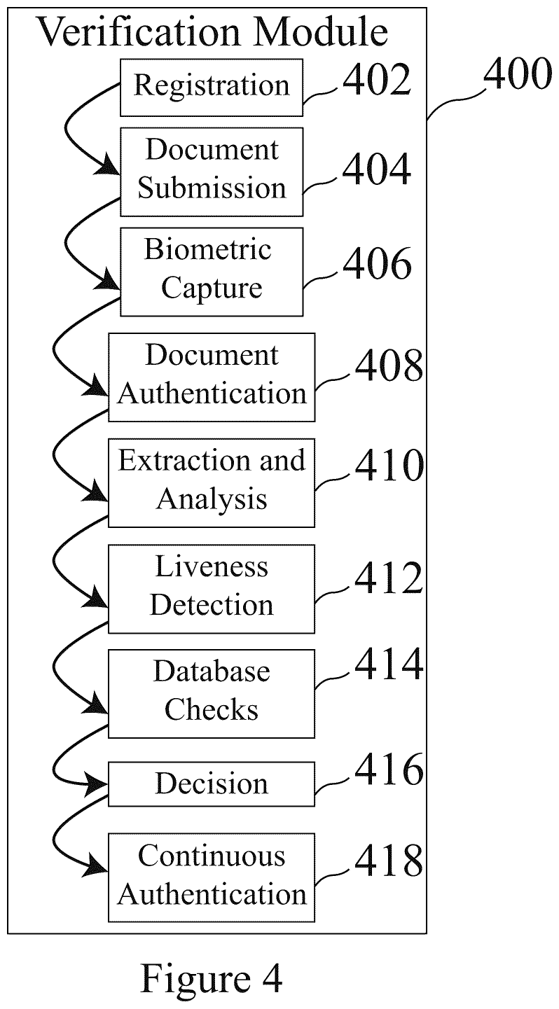

Referring to FIG. 4, there is shown an example of a verification module 400. Verification module 400 may be implemented in place of verification module 104 as shown in FIG. 1. Verification module 400 may include a registration module 402, in which the consumer may initiate a process to create, or log into, an account. The account may then be used to initiate, or be a part of, a verification process in a platform. Examples of a platform may include a company platform, an electronic portal, an identity verification platform of a third party, etc. Registration 402 may be followed by a document submission module 404, in which the consumer may be asked to submit copies of one or more identification documents (e.g., government-issued IDs). The one or more identification documents may be foundational documents for verifying the identity of the consumer. Optionally, a biometric capture 406 (e.g., fingerprints, facial recognition, iris scans, etc.) may be obtained through a device (e.g., a mobile device's camera, a fingerprint scanner, etc.) as an additional means of verifying the consumer's identity. For instance, biometric capture 406 may be cross-referenced with submitted identification documents from document submission module 404 to ensure that the consumer presenting the identification documents is the same consumer as the holder of the identification documents. The identification documents may be authenticated in a document authentication module 408. Document authentication module 408 may use various checks to confirm validity of the identification documents. One of those checks may include checking the identification documents for features which indicate authenticity, such as watermarks, holograms, and/or machine-readable zones. Information from the submitted documents may be extracted via an extraction and analysis module 410. Extraction and analysis module 410 may employ optical character recognition (OCR) to analyze and match provided details. Extraction and analysis module 410 extract text and/or images of the submitted documents. A liveness detection module 412 may be used to ensure that submitted biometrics are from a live person than, for instance, a photo or a video. The consumer may be asked in liveness detection module 412 to perform specific actions during a biometric capture process, such as blinking or moving their head a certain way. A database checks module 414 may employ one or more checks or searches to compare information of databases (e.g., criminal records, watchlists, credit databases, etc.) with information submitted by the consumer to verification module 400. Access to certain databases for database checks module 414 may depend on jurisdiction and the extent of verification required. Based on the outcome of, for example, document authentication, biometric verification, and/or database checks, a verification module 416 may determine whether the consumer's identity is verified. If verified, the consumer may proceed to participate with the system of which verification module 400 is a part of. Verification module 416 may compare the outcome of prior processes with a predetermined set of rules or criteria to determine whether a consumer's identity deserves verification. Verification module 400 may include a continuous, or periodic, authentication module 418 to re-verify a previously verified consumer. Continuous authentication module may use biometrics or other methods to ensure the consumer accessing the system housing verification module 400 over time remains to be the authorized user of the system. Verification module 400 may further include an identity verification application programming interface (not shown) to facilitate movement of consumer information to an identity verification platform (not shown).

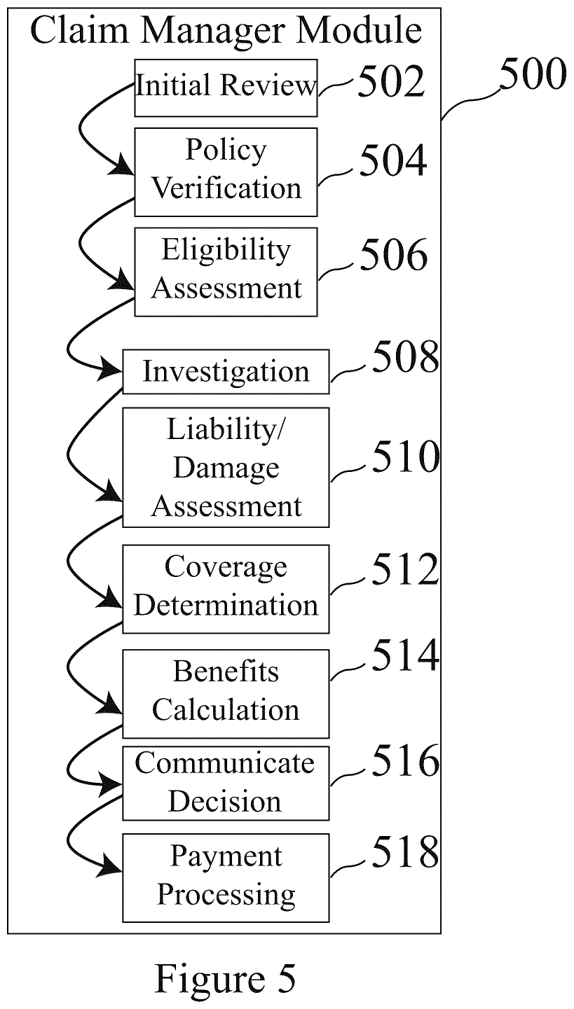

Referring to FIG. 5, there is shown an example of a claim manager module 500. Claim manager module 500 may be implemented in place of claim manager module 146 as shown in FIG. 1. Generated claim 130 may be submitted to claim manager module 500. Claim manager module 500 may be used to verify potential claims. Claim manager module may involve several steps designed to ensure that the potential claim is valid, accurate, and falls within the coverage of the consumer's policy. Claim manager module 500 may begin one or more steps upon receiving a submitted claim. The submitted claim may include all relevant details, including personal information, policy number, and specifics about one or more incidents or services which led to the claim. An initial review module 502 may be employed to conduct an initial review of the claim to ensure the claim submission is complete and contains all necessary information or documentation for its evaluation. Initial review module 502 may request and/or use reports, invoices or proof of loss/damage. A policy verification module 504 may verify policy details (e.g., via a submitted policy or the policy number). Policy verification module 504 may verify various details, including coverage limits, deductibles, exclusions, specific terms which may affect claim eligibility, etc. An eligibility assessment module 506 may, based on the policy of concern, assess whether the submitted claim falls within the scope of the policy's coverage and if the claimant is eligible for benefits under the policy. Eligibility assessment module 506 may implement a large language model, a claim adjuster, a rule-based automation system, a checklist, a standard operating procedure, a database lookup, external verification services, collaboration with a third-party, or peer review. Claim manager module 500 may include an investigation module 508, wherein one or more investigative steps pertinent to a claim (e.g., analysis of additional documentation, interviewing witnesses, consulting experts, or inspecting damages firsthand to verify the claim's accuracy and legitimacy) may be used to verify the submitted claim's accuracy or legitimacy. Assessment of a certain liability or a certain claim to damages may occur via a liability/damage assessment module 510. Liability/damage assessment module 510 may include, for example, reviewing estimates for repairs, replacement costs, medical expenses, etc. Liability/damage assessment module 510 may use the same one or more investigate steps as investigation module 508 for determining the extent of damages or costs associated with the submitted claim. With information and assessments at hand, a coverage determination module 512 may be implemented to decide whether the claim will be approved, partially approved, or denied based on the policy terms and the findings of investigation module 508. Coverage determination module 512 may consider information from large language model 124. Coverage determination module 512 may also, for example, consider deductibles, coverage limits, or any co-insurance requirements. A benefits calculation module 514 may, for an approved or a partially approved claim, calculate the amount of benefits or compensation that the claimant is entitled to receive. Claim manager module 500 may communicate to the consumer/claimant a decision of coverage determination module 512 via a module to communicate the decision 516. Payment processing may occur by claim manager module 500 initiating a payment processing module 518. Payment processing module 518 may include disbursing an agreed-upon benefit to the claimant/consumer or to a relevant service provider.

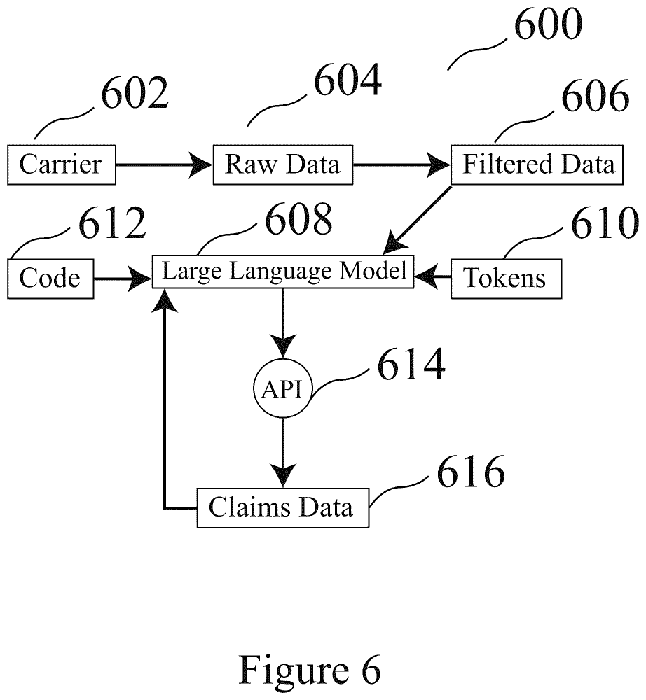

Referring to FIG. 6, there is shown an example of a large language model architecture 600. Large language model architecture 600 may be a part of large language model 124 of FIG. 1. Large language model architecture 600 may include a carrier 602. Carrier 602 may substantially resemble carrier 132 in FIG. 1. Carrier 602 may comprise a wide variety of raw data 604. For example, the raw data may include personal information, policy data (e.g., policy numbers, policy types, coverage details, premium payment history, etc.), claims data (e.g., detailed records of claims submitted, documentation supporting claims, claims processing notes, claims history, etc.), health data (e.g., medical histories, records of physician visits and hospital stays, prescription drug histories, laboratory and imaging results, etc.), property data (e.g., vehicles, property addresses, etc.), financial data (e.g., billing information, records of premium adjustments and refunds, creditworthiness and credit scores, etc.), communication records (e.g., call logs, electronic correspondence, notes from customer service interactions, etc.), telematics data (e.g., data collected from in-vehicle devices, data collected from wearable devices, etc.), and/or web and app usage (e.g., interactions with insurer's website and/or mobile application). Raw data 604 may be generated from or by carrier 602 to be readable by, for example, large language model 124. Large language model 124, or any other large language model implementing large language model architecture 600, may ingest raw data 604 and filter raw data 604 to filter data and yield relevant, filtered data 606. Filtered data 606 may differ from raw data 604 in that it may contain applicable benefits, applicable claims, applicable contract versions of contracts, insured lives, and/or other relevant data which would facilitate large language model processing for generating claims and/or generating claims data. Filtered data 606 may then be acted upon by a large language model 608. Large language model 608 may substantially resemble large language model 124, be a sub-model within large language model 124, or be a large language model independent of large language model 124. Large language model 608, in addition to being able to receive or process filtered data 606, may receive or process tokens 610 and/or a code 612. Tokens 610 may include basic units of data to be processed by large language model 608. Tokens 610 may come from other sources outside of large language model architecture 600. Tokens 610 may be words, parts of words (like syllables or subwords), or even individual characters, depending on a tokenization technique used. Tokenization may be a process of converting the input text into a sequence of tokens that large language model 608 may understand. For example, during training of large language model 608, text data may first be tokenized. Each token may then convert into a numerical representation, e.g., through embeddings that capture semantic information about the token. Large language model 608 may learn to predict the next token in a sequence given the previous tokens, adjusting its internal parameters based on the error in its predictions during a training process. This learning may involve processing vast amounts of tokenized text, enabling the model to understand language patterns, grammar, context, and even generate coherent text based on the tokens it receives as input. Code 612 may be a healthcare code or a medical code derived from a healthcare code system or a medical code system. Code 612 may include at least one ICD/CPT/HCPCS/LOINC/NDC code (International Classification of Diseases/Current Procedural Terminology/Healthcare Common Procedure Coding System/Logical Observation Identifiers Names and Codes/National Drug Code). Code 612 may be used to train large language model 608. Large language model 608 may generate claims data 616 through an application programming interface layer (API layer) 614. API layer 614 may orchestrate output of large language model 608 to create claims data which may be readable by a computer program or a human being. Claims data 616 may be fed into large language model 608 for further training. Claims data 616 may include adjudicated data for paid claims or potentially paid claim decisions. Claims data 616 may be generated by large language model 608 or be a combination of large language model 608 output and other information sourced from outside large language model architecture 600 (not shown). Large language model architecture may use an application programming interface (not shown) to collect carrier 602 data or provide benefits, claims, contract, or insured lives data from carrier 602 to a large language model such as large language model 124 of system 100.

Large language models 124 or 608 may function by processing text as a series of tokens, where a token can be a word, part of a word, or even a character, depending on the tokenization scheme used. Large language models 124 or 608 may use subword tokenization schemes (e.g., Byte Pair Encoding (BPE), SentencePiece, WordPiece, etc.) to handle vocabulary. The subword schemes may include splitting words into smaller units (e.g., subwords or characters) which can represent both common words as whole tokens and rare words as combinations of subtokens, reducing the vocabulary size and handling out-of-vocabulary words better. To account for order of words in text, transformer-based models may add positional encodings to input embeddings. These encodings may be vectors which represent each token's position in a sequence, ensuring, for example, that large language models 124 or 608 can consider word order despite a transformer architecture's non-sequential processing nature. Transformer architecture may include a self-attention mechanism, which would allow each token (e.g., of tokens 610) to interact with every other token in an input sequence as weighted by their relevant to a current token (e.g., a current word, subword, or character(s)). The mechanism may include a quantifying process, wherein attention scores (e.g., calculating using query, key, and value vectors derived from input embeddings) may be used to enable large language models 124 or 608 to focus on relevant parts of an input when making predictions. Large language models 124 or 608 may use multiple sets of vectors to simultaneously focus on different parts of the input. This parallel processing of different attention means may enrich each of the large language model's understanding of context. In an instance where large language models 124 or 608 generate text, they may use decoding strategies (e.g., greedy decoding, beam search, top-k sampling, etc.) to select tokens (e.g., tokens 610) step by step. A temperature parameter may be implemented to control randomness in token selection. Statistical mechanisms may be integrated into large language models 124 or 608 to measure differences between predicted probabilities of an upcoming token and an actual token implemented in data used by either large language model 124 or 608.

### GENERAL

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

A number of implementations have been described. Having thus described the disclosure of the present application in detail and by reference to embodiments thereof, it will be apparent that modifications and variations are possible without departing from the scope of the disclosure defined in the appended claims.

What is claimed is:

1. A system for processing a claim through an automatic submission of the claim on behalf of a consumer, the system comprising:
An electronic interface comprising computer-readable media;
a consumer opt-in module implemented by the electronic interface;
a consumer verification module implemented by the electronic interface and configured to employ at least one of multi-factor authentication, digital certificates, digital signatures, and biometric verification to verify the identity of the consumer;
a policy stored in the computer-readable media;
a consumer electronic health record stored in the computer-readable media;
an electronic data feed comprising real-time claim adjudication data and real-time monitoring data;
a code catalogue comprising a first code and a second code;
a notification module implemented within the electronic interface;
an application programming interface implemented by the electronic interface and configured to facilitate access to data of the consumer electronic health record through an authentication module that employs at least one of API keys and OAuth tokens to ensure that only authorized users or entities can access the data of the consumer electronic health record;
a large language model trained on electronic health records and medical coding standards and implemented by the electronic interface and configured to
process natural language processing techniques that extract clinical entities, match them to standardized medical terminology, and identify relevant procedure and diagnostic codes,
interact with the application programming interface,
obtain the consumer electronic health record,
read data of the consumer electronic health record,
assign the first code to the data of the consumer electronic health record based on the consumer electronic health record's contents,
read the electronic data feed,
listen for medical events from the electronic data feed by matching codes with consumer information,
associate the data of the consumer electronic health record and the assigned code with one or more aspects of the electronic data feed to create an associated data set,
read the policy,
perform statistical analyses to compare the associated data set with the policy,
generate an editable claim based on the comparison between the associated data set and the policy,
assign the second code to the generated editable claim,
generate a notification via the notification module based on the second code, and
notify whether the generated editable claim is accepted, under review, or denied;
a computer-readable database implemented by the electronic interface and configured to receive queries, updates, and validations from the large language model; and
an error detection module implemented by the electronic interface and configured to monitor the electronic data feed, identify errors based on predefined significance thresholds, and release an error notification.

2. The system of claim 1, wherein the application programming interface further comprises an authentication module.

3. The system of claim 1, further comprising an input form.

4. The system of claim 1, further comprising a claim manager module.

5. The system of claim 4, wherein the claim manager module is configured to generate the input form when the generated editable claim is under review.

6. The system of claim 4, wherein in the event the generated editable claim is denied, the generated editable claim is automatically sent to the claim manager module.

7. The system of claim 1, wherein the generated editable claim is configured to be editable by an individual.

8. The system of claim 2, wherein the database further comprises a code library and a health data standard, wherein the code library is configured to interact with the health data standard and wherein the large language model is configured to access and employ the code library.

9. The system of claim 1, wherein the electronic data feed further comprises claim adjudication data.

10. The system of claim 1, further comprising an error detection module configured to monitor the electronic data feed, identify errors, and release an error notification; and a control module configured to receive the error notification.

11. A method for processing a claim through an electronic interface and an automatic submission of the claim on behalf of a consumer, the method comprising:
opting in a consumer through a consumer opt-in module to authorize automatic data extraction, claim submission, a payment method, and a communication method;
verifying the identity of the consumer through multi-factor authentication, digital certificates, digital signatures, or biometric verification;
obtaining a policy of the consumer;
obtaining the consumer's electronic health record;
providing an electronic data feed comprising claim adjudication data;
providing a code catalogue comprising a first code and a second code, wherein the first code and the second code are each a healthcare code or a medical code;
providing a notification module;
providing an application programming interface configured to facilitate access to consumer's electronic health record through an authentication module that employs at least one of API keys and OAuth tokens;
providing a large language model configured to
interact with the application programming interface,
obtain the consumer electronic health record,
read data of the consumer electronic health record,
assign the first code to the data of the consumer electronic health record based on the consumer electronic health record's contents,
read the electronic data feed,
listen for medical events by matching codes with consumer information,
associate the data of the consumer electronic health record and the assigned code with one or more aspects of the electronic data feed to create an associated data set,
read the policy,
compare the associated data set with the policy using statistical analyses,
generate an editable claim based on the comparison between the associated data set and the policy,
assign the second code to the generated editable claim,
generate a notification via the notification module based on the second code, and
notify whether the generated editable claim is accepted, under review, or denied;
sending queries, updates, and validations to a database;
classifying the claim as payable or potentially payable; and
monitoring the electronic data feed, identifying errors, and releasing an error notification.

12. The method of claim 11, further comprising sending the generated editable claim to a claim manager module.

13. The method of claim 12, further comprising investigating the generated editable claim and notifying the consumer of investigation details.

14. The method of claim 11, further comprising obtaining raw data from a carrier.

15. The method of claim 14, further comprising filtering the raw data.

16. The method of claim 15, further comprising generating claims data arranged to be fed into the large language model.

17. The method of claim 11, further comprising obtaining biometric data of the consumer.

18. The method of claim 11, wherein the first code and the second code are each a healthcare code or a medical code.

19. The method of claim 11, further comprising extracting health data of the consumer after generating a claim.

20. The method of claim 11, further comprising classifying the claim as payable or potentially payable.
